(19) United States
(12) Patent Application Publication
(10) Pub. No.: US 2022/0280556 A1
(43) Pub. Date: Sep. 8, 2022

(54) METHOD AND USE OF COMPOSITIONS COMPRISING LIGNOSULFONATE FOR ATTENUATION OF SARS-CoV-2 VIRUS REPLICATION

(71) Applicant: Attenubiotics Inc., Calgary (CA)

(72) Inventor: Ralph Kevin Walker, Calgary (CA)

(21) Appl. No.: 17/689,856

(22) Filed: Mar. 8, 2022

Related U.S. Application Data
(60) Provisional application No. 63/158,271, filed on Mar. 8, 2021.

(51) Int. Cl. A61K 31/737

(52) U.S. Cl. CPC A61K 31/737; A23K 10/30

(57) ABSTRACT

The use of compositions comprising lignosulfonate for attenuation of SARS-CoV-2 virus replication. The compositions can be used in methods for preventing and treating COVID-19 and other medical disorders caused by or associated with SARS-CoV-2 virus replication in humans and animals. In some embodiments the compositions are substantially free of elemental sulphur. In some embodiments the lignosulfonate is radically polymerized. In some embodiments the compositions are formulated as an animal feed additive or supplement. The disclosure encompasses compositions comprising lignosulfonate and methods of preventing or treating COVID-19 by administering compositions comprising lignosulfonate to human or animal subjects in an effective dose to attenuate the pathogenic effect of the SARS-CoV-2 virus.

### CROSS-REFERENCE TO RELATED APPLICATIONS

This application claims the benefit under 35 U.S.C. § 119 of U.S. application No. 63/158,271 filed 8 Mar. 2021 and entitled METHOD AND USE OF COMPOSITIONS COMPRISING LIGNOSULFONATE FOR ATTENUATION OF SARS-CoV-2 VIRUS REPLICATION, which is hereby incorporated herein by reference for all purposes.

### TECHNICAL FIELD

This application relates to the use of compositions comprising lignosulfonate for attenuation of severe acute respiratory syndrome coronavirus 2 (SARS-CoV-2) virus replication. In some embodiments the compositions can be used in methods for preventing and treating COVID-19 and other medical disorders caused by or associated with SARS-CoV-2 virus replication in humans and animals.

### BACKGROUND

Many medical disorders are caused by pathogenic agents such as bacteria or viruses. Conventional treatment of such disorders is focused on killing or removing the pathogen, such as by administering antibiotics or anti-viral drugs. However, such treatment is often not effective, for example due to bacterial or viral resistance.

Compositions intended to attenuate the effect of pathogenic agents are known in the prior art. For example, United States Patent Application Publication No. US 2015/0132390 A1, which is incorporated by reference herein in its entirety, describes a pharmaceutical preparation comprising as an active ingredient micron-sized sulphur particles. The preparation may also include sodium lignin sulphate (sometimes referred to as sodium lignosulfonate). As described in the '390 publication, the purpose of the sulphur particles is not to kill a target pathogen but rather to re-establish a healthy equilibrium or homeostasis from a pathological imbalance or disorder. According to the theory of the '390 disclosure, the compound is believed to act by depriving the pathogen of oxygen with available sulphur atoms that are oxidized. This results in a “pathogenic attenuation”, namely a slowing down or decrease in the rate of reproduction of the pathogen. Such attenuation enables the host's natural defenses, such as the host's immune system, to more quickly and effectively eliminate or control the pathogen. Moreover, attenuation of pathogens may result in less prolonged activation of the host's defenses which may improve the overall health and physiological performance of the animal.

Patents and patent applications related to the '390 publication have been granted and/or published as follows: AU20072572862; CA265408C; CN101500584B; EP2035018B1; JP2009538837A; JP2013231072A; KR10146209B1; MX2008015200A; and NZ597657. All of the above patents and publications are incorporated by reference herein in their entirety.

The '390 publication and related patents and/or publications identify elemental sulphur as the active therapeutic ingredient. Sodium lignin sulphonate is described as acting as a catalyst for removing the sulphur atoms from a ring structure thereby making the sulphur atoms available for systemic oxidization and a corresponding reduction in the production of systemic hydrogen protons. However, the inventor of the present disclosure has determined that compositions comprising lignosulfonate are biologically effective for use in pathogenic attenuation even in the absence of elemental sulphur.

Co-pending U.S. patent application Ser. No. 16/523,901 filed 26 Jul. 2019, published under No. US-2020-0054670 on 20 Feb. 2020, which is incorporated by reference herein in its entirety, is directed to compositions which comprise lignosulfonate and are substantially free of elemental sulphur for pathogenic attenuation. Pathogens of particular medical interest are SARS-related coronaviruses including severe acute respiratory syndrome coronavirus 2 (SARS-CoV-2). This virus is the cause of the disease COVID-19 which is presently the subject of a worldwide pandemic. Novel therapies for preventing or treating COVID-19 by attenuating SARS-CoV-2 virus replication are urgently needed. The present application is directed to methods of attenuating of SARS-CoV-2 virus replication using compositions comprising lignosulfonate for treatment of COVID-19 or other medical disorders in humans and animals.

The foregoing examples of the related art and limitations related thereto are intended to be illustrative and not exclusive. Other limitations of the related art will become apparent to those of skill in the art upon a reading of the specification and a study of the drawing(s).

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In one aspect, this application relates to the use of a composition comprising lignosulfonate for attenuation of severe acute respiratory syndrome coronavirus 2 (SARS-CoV-2) virus replication. In some embodiments the lignosulfonate is substantially free of elemental sulphur.

In another aspect, this application relates to a method of preventing or treating COVID-19 in a human or animal subject comprising administering to the subject an effective amount of a composition comprising lignosulfonate. In some embodiments the lignosulfonate is substantially free of elemental sulphur.

In another aspect, this application relates to a pharmaceutical composition comprising lignosulfonate, wherein the composition is formulated in a dosage for use in the prevention or treatment of COVID-19 in a human or animal subject. In some embodiments the lignosulfonate is substantially free of elemental sulphur.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced FIGURES of the drawing(s). It is intended that the embodiments and FIGURE(s) disclosed herein are to be considered illustrative rather than restrictive.

FIG. 1 is a graph plotting in vitro cell viability and virus titer levels relative to varying concentrations of a lignosulfonate test composition. Each data point represents an average of four replicates.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The present application is directed to uses, methods and compositions comprising lignosulfonates for attenuation of SARS-CoV-2 viral replication. As used in the present application “attenuation” means reduction or cessation of virus replication. Without being bound by any particular theory, the inventor believes that such attenuation may enable the host animal to mount an effective immune or other biological response to a viral pathogen. For example, the compositions may prevent the pathogen population from growing exponentially in the host animal, thereby enabling the animal to mount an effective immune or other biological response.

In some embodiments the compositions can be used in methods for preventing and treating COVID-19 and other medical disorders caused by or associated with SARS-CoV-2 virus replication in humans and animals. In other embodiments the compositions can be used for the attenuation of other viral pathogens such as other SARS-related coronaviruses and in methods for preventing and treating medical disorders caused by such other viral pathogens.

Along with cellulose, lignin is one of the primary constituents providing the structure of wood. Lignosulfonates are amorphous branched polymers of lignin resulting from the sulfite pulping process used to delignify wood or other lignocellulosic biomasses. Lignosulfonates may contain sulfonated covalently linked phenyl propane monomers and other heterogeneous compounds. In some embodiments lignosulfonates may be in the form of ammonium lignosulfonate, sodium lignosulfonate, calcium lignosulfonate and magnesium lignosulfonate.

Lignosulfonates are commonly used for many different applications, including as binders, pelletizing agents, briquetting agents, anti-caking agents, surfactants, dust suppressants and coagulants. Lignosulfonates are also used as macronutrients in feedingstuffs for animals, including cattle, pigs and chickens. Toxicological studies on lignosulfonates indicate that they are non-toxic. In the literature, the LD50 for lignosulfonates has been reported to be 20,000 mg/kg body weight be ingestion. Materials with LD50 values of 5,000 mg/kg of body weight or greater are considered to be non-toxic (e.g. Material Safety Data Sheet, Ammonium lignosulfonate liquid, Tembec—Chemical Group, accessible online at www.maritimehydroseed.com/images/TDS_MSDS.pdf). Further, lignosulfonates have been approved by the USDA for inclusion in animal feeds.

In some embodiments the lignosulfonates may be substantially free of elemental sulphur. In some embodiments the lignosulfonates may be radically polymerized. For example, the lignosulfonates may be radically polymerized by subjecting the lignosulfonates to very high heat.

Compositions comprising lignosulfonates are generally available from a wide variety of sources. By way of an exemplary example, a composition comprising radically polymerized ammonium lignosulfonates is or has been available from Rayonier Advanced Materials of Montreal, Quebec, and has been sold under the trademark ARBO® ARBO-FEED (hereinafter “ARBO-FEED”). A chemical analysis of ARBO-FEED in powder form has confirmed that it does not include a measurable amount of elemental sulphur. In particular, the ARBO-FEED composition contained less than the reportable detection limit of sulphur (elemental) in a chemoanalytic analysis where the detection limit was 100 mg/kg.

The ARBO-FEED composition comprising radically polymerized lignosulfonates may also comprise measurable amounts of tannic acid and aldehydes such as formaldehyde, acetaldehyde and propionaldehyde. Without being bound by any particular theory, the inventor believes that one or more of the above constituents could provide a biological effect in vivo, either directly or acting synergistically with other multi-molecular constituents. Further, without being bound by any particular theory, the inventor believes that the ARBO-FEED composition comprising radically polymerized lignosulfonates may function as an ATP inhibitor, likely via ATPase enzymes embedded in the mitochondrial membrane, to effect attenuation SARS-CoV-2 virus replication at biologically effective dosages.

### Example 1

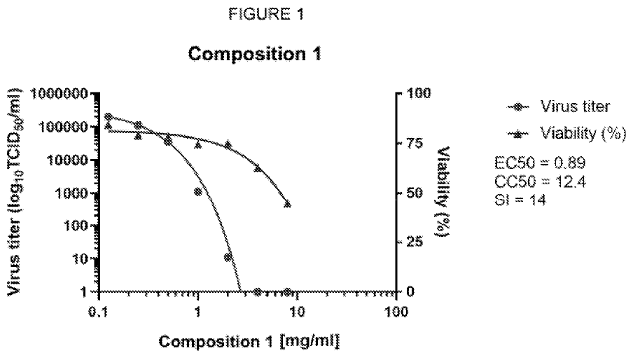

The objective of the study of Example 1 was to determine the in vitro antiviral activities of a lignosulfonate test composition consisting of ARBO-FEED (identified as Composition 1 in FIG. 1). The antiviral activities were measured in respect of severe acute respiratory syndrome coronavirus 2 (SARS-CoV-2). The test composition was provided in powdered form and was dissolved in sterile Minimum Essential Medium (Eagle) to the desired concentration.

According to the method of this study, Vero'76 cells were seeded and grown overnight at 37° C. in a 5% CO₂ environment to approximately 90% confluency in 96-well plates. The culture medium used was Dulbecco's Modified Eagle Medium (DMEM) supplemented with 10% fetal bovine serum (FBS) and antibiotics penicillin and streptomycin (PEN-STREP).

The SARS-CoV-2 virus (SARS-CoV-2/Canada/ON/VIDO-01/2020/Vero'76/p.2) was diluted in DMEM supplemented with 2% FBS/PEN-STREP to obtain a multiplicity of infection (MOI) of 0.1 (approximately 2000 TCID₅₀/well). The culture medium was removed from the cells, and 50 μl of the virus inoculum was added to each well. The plates were incubated for 1 hour at 37° C. in a 5% CO₂ environment.

In this example, the concentration ranges of the test composition were 0.125-8.00 mg/ml. The test composition was dissolved and 2-fold serial dilutions were made using DMEM supplemented with 2% FBS/PEN-STREP. After the viral infection plates had been incubated for 1 hour, the medium was removed from each well. The diluted test solution (100 μl) was then added to the infected cells. The assay was done in quadruplicate. Medium alone was used as a control for virus replication, and cell-alone control wells (without viral infection and without test composition) were also established. A separate plate was used to assess the cell toxicity of the test composition by incubating uninfected cells with the serially diluted test composition solution. The assays were performed in 4-replicates in a 96-well plate.

After 48 hours of incubation, wells of the plate used for testing of cytotoxicity of the test composition were treated with CellTiter 96 Aqueous One Solution Reagent (Promega, Cat #G3580). The plate was then incubated for 2 hours at room temperature, and then the absorbance at 490 nm was determined using a microplate reader. The corrected absorbance at 490 nm versus concentration of growth factor was plotted to determine cytotoxic concentration. Cell viability of untreated cells was set at 100% and was based on the absorbances of wells containing cell only plus media and solvent (after correction for the background).

The plate of infected cells, which had been exposed to the test composition was examined for cytotoxicity/contamination at 24 hours, and cytopathic effect (CPE) under microscope at 48 hours. At 48 hours, the cells from each well were harvested and washed with phosphate-buffered saline (PBS). The supernatant from each well was also harvested. Viral titration by the TCID₅₀ assay of the supernatant was carried out by a serial 10-fold dilution. These dilutions were used to infect pre-seeded cells, in quadruplicates as described for the initial infection. Cells were observed for CPE at 1, 3 and 5 days after the infection. The Median Tissue Culture Infectious Dose (TCID₅₀) was calculated according to the method of Spearman & Karber:

https://www.ncbi.nlm.nih.gov/pmc/articles/PMC4861875/.

The selectivity index (SI) is a ratio that measures the window between cytotoxicity and antiviral activity and is determined by dividing antiviral activity (effective concentration (EC50)) by the cytotoxicity (50% cytotoxic concentration (CC50)). The EC50 and CC50 were calculated using Compusyn software (https://www.combosyn.com/).

The results of the study in respect of the lignosulfonate test composition (Composition 1) are summarized in Table 1 below

TABLE 1

| Concentration of lignosulfonate test | Virus titer | Cell Viability |
|---|---|---|
| composition (Composition 1) (mg/ml) | (TCID₅₀)* | (%) |
| 8 | 0 | 40.31 |
| 4 | 0 | 56.76 |
| 2 | 11.2 | 67.79 |
| 1 | 1120 | 67.46 |
| 0.5 | 35600 | 70.80 |
| 0.25 | 112000 | 71.32 |
| 0.125 | 200000 | 76.22 |
| 0 | 112000 | 100 |

*TCID50 titer calculated by Spearman & Karber method

Antiviral activities of the lignosulfonate test composition are shown in FIG. 1. The TCID₅₀ titer was 1.12×10⁵ without treatment. Treatment with the test composition at a concentration of 4 mg/ml or greater resulted in no virus being detected (since FIG. 1 consists of a logarithmic scale the absence of virus was plotted at the lowest virus titer shown). Treatment with a concentration of 2 mg/ml resulted in a TCID₅₀ titer of to 1.12×10¹, a four log reduction in titer. As shown in FIG. 1, the calculated EC50 was 0.89, the CC50 was 12.4 and the SI was 14.

The cell viability (%) for the control well on the viability plate (cells and medium only) was set as 100%. Cell viability was about 76% at a concentration of 0.125 mg/ml and slowly dropped to about 68% at a concentration of 2 mg/ml. At greater concentrations of test composition the cell viability decreased significantly (FIG. 1). As will be apparent to a person skilled in the art, viral replication requires viable cells. Accordingly, viral titer measurements at low cell viability concentrations may not be indicative of viral replication inhibition. With reference to Table 1, concentrations of the lignosulfonate test composition in a range between about 0.5 to 2 mg/ml resulted in a relatively low virus titer while maintaining a relatively high cell viability, demonstrating likely in vitro attenuation of SARS-CoV-2 virus replication.

### Example 2

The objective of the study of Example 2 was to determine the therapeutic effect of lignosulfonate in hamsters challenged with SARS-CoV-2. The lignosulfonate was given in drinking water to Syrian golden hamsters 4 weeks prior to challenge with SARS-CoV-2. One week after challenge, blood was collected from the hamsters and blood serum was analyzed to assess the concentration of various cytokines in the serum samples.

According to the method of this study, lignosulfonate, namely a lignosulfonate test composition consisting of ARBO-FEED as described in Example 1 above, was given in drinking water to the hamsters at 3 different test concentrations, namely 0.5 mg/ml, 1.0 mg/ml and 2.0 mg/ml. Drinking water was ad libitum expecting the consumption to be proportional to body weight of the hamsters. The expected daily dose for the group given 0.5 mg/ml is 42.5 mg/kg; for the group given 1 mg/ml is 85 mg/kg; and for the group given 2 mg/ml is 170 mg/kg. Hamsters were treated with lignosulfonate in the water for 4 weeks (28 days) and then infected with SARS-CoV-2 intranasally. The SARS-CoV-2 virus was as described in Example 1 above. The effect of lignosulfonate against SARS-CoV-2 was assessed by quantification of cytokines in the serum by ELISA assay, as discussed below.

The treatment group and doses for the trial was as set out in Table 2 below:

TABLE 2

| | | | Dailydose | | Volume daily |
|---|---|---|---|---|---|
| Group | N | Treatment | (mg/kg) | Route | (ml) |
| A | 6 | None | N. A. | In drinking | 8.5 ml per 100 g |
| | | | | water | body weight |
| B | 6 | Lignosulfonate | 42.5 mg/kg | In drinking | 8.5 ml per 100 g |
| | | at 0.5 mg/ml | for 35 days | water | body weight |
| C | 6 | Lignosulfonate | 85.0 mg/kg | In drinking | 8.5 ml per 100 g |
| | | at 1.0 mg/ml | for 35 days | water | body weight |
| D | 6 | Lignosulfonate | 170.0 mg/kg | In drinking | 8.5 ml per 100 g |
| | | at 2.0 mg/ml | for 35 days | water | body weight |

The average water consumption per animal for the pre-challenge period was 7.22 ml/day for Group A; 6.70 ml/day for Group B; 7.71 ml/day for Group C; and 6.10 ml/day for Group D. The higher consumption of groups A and C may be due to some loss of liquid from the drinking pouch, because the uptake for these data points was significantly higher on one occasion for each group.

Blood was collected from the hamsters one week (7 days) post-infection. The serum was separated from the blood and the following cytokines were analyzed by ELISA assay: IL-6, IL-17, IL-10, TGF-β, IL-4, IL-12, IL-2, TNF-α, IFN-γ, IL-1B and IL-1.

The ELISA assay results showed significant differences between the lignosulfonate treated and the control untreated hamsters (Group A) in respect of cytokines IL-12 and IL-1β. No statistically significant differences were detected between the lignosulfonate treated and the control untreated hamsters (Group A) in respect of the other cytokines tested.

More particularly, hamsters treated with lignosulfonates at dose 170 mg/kg (Group D) showed significant increase in IL-1β (p<0.05) compared to the control untreated group. Further, hamsters treated with lignosulfonates at dose 42.5 mg/kg (Group B) showed significant increase in IL-12 (p<0.05) compared to the control untreated group (Group A) and hamsters treated with lignosulfonates at dose 170 mg/kg (Group D) showed significant decrease in IL-12 (p<0.01) compared to the 42.5 mg/kg (Group B) group.

Both cytokines IL-12 and IL-1β are known to have immunomodulatory effects. The interrelationship between IL-12 and IFN-γ is particularly complex. Without being bound by any particular theory, the inventor postulates that lignosulfonate activates at least cytokine IL-12, and IL-12 is a potent inducer of IFN-γ production. Although IL-12 has other direct effects on T and NK cells, e.g, acting as a growth factor and an enhancer of cytotoxicity as well as inducing other cytokines, many of the effects of IL-12 have been attributed to its ability to induce production of IFN-γ. IFN-γ produced by T and NK cells in response to IL-12 acts as a powerful positive feedback mechanism on the phagocytic cells producing IL-12, activating them and enhancing their ability to produce many pro-inflammatory cytokines, including IL-12 itself and IFN-γ. It is believed that such immunomodulatory effects may play a role in attenuation of severe acute respiratory syndrome coronavirus 2 (SARS-CoV-2) virus replication.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. Use of a composition comprising lignosulfonate for attenuation of severe acute respiratory syndrome coronavirus 2 (SARS-CoV-2) virus replication.

2. The use as defined in claim 1, wherein said composition is substantially free of elemental sulphur.

3. The use as defined in claim 1, wherein said lignosulfonate is radically polymerized lignosulfonate.

4. The use as defined in claim 1, wherein said composition comprises lignosulfonate selected from the group consisting of ammonium lignosulfonate, sodium lignosulfonate, calcium lignosulfonate and magnesium lignosulfonate.

5. The use as defined in claim 1, wherein said composition is formulated for use as an animal feed additive or supplement.

6. The use as defined in claim 1, wherein said composition is administered in a dosage of between 0.0125 and 2 mg/ml.

7. The use as defined in claim 6, wherein said composition is administered in a dosage of between 0.5 and 2 mg/ml.

8. The use as defined in claim 7, wherein said composition is administered in a dosage of between 0.5 and 1 mg/ml.

9. The use as defined in claim 1, comprising administering said composition to a human or animal subject.

10. A method of preventing or treating COVID-19 in a human or animal subject comprising administering to said subject an effective amount of a composition comprising lignosulfonate.

11. The method as defined in claim 10, wherein said composition is substantially free of elemental sulphur.

12. The method as defined in claim 10, wherein said lignosulfonate is radically polymerized lignosulfonate.

13. The method as defined in claim 10, wherein said composition comprises lignosulfonate selected from the group consisting of ammonium lignosulfonate, sodium lignosulfonate, calcium lignosulfonate and magnesium lignosulfonate.

14. The method as defined in claim 10, wherein said composition is formulated for administration as an animal feed additive.

15. The method as defined in claim 10, wherein said composition is administered in a dosage of between 0.0125 and 2 mg/ml.

16. The method as defined in claim 15, wherein said composition is administered in a dosage of between 0.5 and 2 mg/ml.

17. The method as defined in claim 16, wherein said composition is administered in a dosage of between 0.5 and 1 mg/ml.

18. The method as defined in claim 10, wherein said composition comprises a pharmaceutically effective carrier, diluent, adjuvant or excipient.

19. The method as defined in claim 10, wherein the selectivity index of said composition is greater than 10.

20. A pharmaceutical composition comprising lignosulfonate formulated in a dosage for use in the prevention or treatment of COVID-19 in a human or animal subject.

21. The composition as defined in claim 20, wherein said composition is substantially free of elemental sulphur.

22. The composition as defined in claim 20, wherein lignosulfonate is radically polymerized lignosulfonate.

23. The composition as defined in claim 20, wherein said dosage of said lignosulfonate is between 0.0125 and 2 mg/ml.

24. The composition as defined in as defined in claim 23, wherein said dosage of said lignosulfonate is between 0.5 and 2 mg/ml.

25. The composition as defined in claim 24, wherein said dosage of said lignosulfonate is between 0.5 and 1 mg/ml.

26. The composition as defined in claim 20, wherein said composition comprises a pharmaceutically effective carrier, diluent, adjuvant or excipient.

27. Use of a composition comprising lignosulfonate for attenuation of a viral pathogen, wherein the viral pathogen is a coronavirus.

28. The use as defined in claim 27, wherein the coronavirus is a severe acute respiratory syndrome (SARS)-related coronavirus.

29. The use as defined in claim 27, wherein the coronavirus is severe acute respiratory syndrome coronavirus 2 (SARS-CoV-2).

30. The use as defined in claim 1, comprising activating one or more of IL-12 and IL-1β.

31. The method as defined in claim 10, comprising activating one or more of IL-12 and IL-1β.

32. The composition as defined in claim 20, wherein the lignosulfonate is formulated in a dosage for activating one or more of IL-12 and IL-1β in the human or animal subject.
